(19) United States
(12) Patent Application Publication
(10) Pub. No.: US 2025/0087312 A1
(43) Pub. Date: Mar. 13, 2025

(54) SYSTEMS AND METHODS FOR PROVIDING A NONPRESCRIPTION DRUG

(71) Applicant: CHATTEM, INC., Bridgewater, NJ (US)

(72) Inventors: Mridul SHAH, Bridgewater, NJ (US); Alankar GUPTA, Bridgewater, NJ (US); Shahper RAHMAN, Bridgewater, NJ (US); Thomas McGRAW, Bridgewater, NJ (US); Lindsey SENTER, Bridgewater, NJ (US); Gina CHARALAMBIDES, Bridgewater, NJ (US); Claudia METCALF, Bridgewater, NJ (US); Paul HOUSEWORTH, Bridgewater, NJ (US); Geoff WYATT, Toronto (CA); Stephane BISSONNETTE, Bridgewater, NJ (US)

(21) Appl. No.: 18/292,646

(22) PCT Filed: Jul. 28, 2021
(86) PCT No.: PCT/US2021/043385

§ 371 Date: Jan. 26, 2024

(51) Int. Cl. G16H 10/20; G16H 20/13; G16H 80/00

(52) U.S. Cl. CPC G16H 10/20; G16H 20/13; G16H 80/00

(57) ABSTRACT

A system may be configured to self-select provision of a nonprescription drug. Some embodiments may: select a mode of implementing a health survey preapproved by a regulatory body; control, via the selected mode and a processor, a sequence of the health survey; determine, via a processor, whether a user undergoing the survey is authorized to purchase the drug; and responsive to the user being authorized, provide a plurality of ways to make the purchase.

### TECHNICAL FIELD

The present disclosure generally relates to systems and methods for implementing a self-selection or self-diagnostic tool that facilitates access and purchase of over the counter (OTC) medication, while maintaining acceptable control over the distribution of the OTC medication.

### BACKGROUND

Users typically purchase over-the-counter medications without a prescription via retail or online. Typically, users will check the OTC package which contains the product labeling (drug facts label (DFL)) to determine if the product is right for them and purchase the product. When users are unable to appropriately self-select the OTC medication with drug facts label alone there is a need to implement a condition of use that allows to check to make sure the OTC medication is appropriate for someone to purchase without having to interact with health care practitioners (HCPs).

### SUMMARY

Systems and methods are disclosed for assisting potentially at-risk consumers (e.g., who currently take certain medications, have a health history, or have underlying conditions) with correct self-selection. Accordingly, one or more aspects of the present disclosure relate to a method for self-selecting provision of a nonprescription drug, via a medical-grade algorithm (e.g., one that is preapproved by a federal agency, such as the Food and Drug Administration (FDA)). Some embodiments may: select a mode of implementing a health survey preapproved by a regulatory body; control, via the selected mode and a processor, a sequence of the health survey; determine, via a processor, whether a user undergoing the survey is authorized to purchase the drug; and responsive to the user being authorized, provide a plurality of ways to make the purchase.

The method is implemented by a system comprising one or more hardware processors configured by machine-readable instructions and/or other components. The system comprises the one or more processors and other components or media, e.g., upon which machine-readable instructions may be executed. Implementations of any of the described techniques and architectures may include a method or process, an apparatus, a device, a machine, a system, or instructions stored on computer-readable storage device(s).

### BRIEF DESCRIPTION OF THE DRAWINGS

The details of particular implementations are set forth in the accompanying drawings and description below. Like reference numerals may refer to like elements throughout the specification. Other features will be apparent from the following description, including the drawings and claims. The drawings, though, are for the purposes of illustration and description only and are not intended as a definition of the limits of the disclosure.

FIG. 1 illustrates an example of a system in which a determination of medical use of a drug is safely self-selected, in accordance with one or more embodiments.

FIGS. 2A-2B illustrate user interfaces (UIs) with questions of a predetermined sequence, in accordance with one or more embodiments.

FIGS. 3A-3B illustrate UIs with health-related attestations of a predetermined sequence, in accordance with one or more embodiments.

FIGS. 4A-4B illustrate UIs with HCP-related questions and attestations of a predetermined sequence, in accordance with one or more embodiments.

FIGS. 6A-6B illustrate front isometric views of a drug-dispensing kiosk, in accordance with one or more embodiments.

FIGS. 5 and 7 illustrate a process for determining means for safely dispensing OTC a drug, in accordance with one or more embodiments.

FIG. 8 is a first UI of a display screen in a sequence of a health survey assessment (HSA) that self-selects users according to a first embodiment:

FIG. 9 is a second UI of the display screen in the sequence according to a first embodiment:

FIG. 10 is a third UI of the display screen in the sequence according to a first embodiment:

FIG. 11 is a fourth UI of the display screen in the sequence according to a first embodiment:

FIG. 12 is a fifth UI of the display screen in the sequence according to a first embodiment:

FIG. 13 is a sixth UI of the display screen in the sequence according to a first embodiment:

FIG. 14 is the first UI of the display screen in the sequence according to a second embodiment:

FIG. 15 is the second UI of the display screen in the sequence according to a second embodiment:

FIG. 16 is the third UI of the display screen in the sequence according to a second embodiment:

FIG. 17 is the fourth UI of the display screen in the sequence according to a second embodiment:

FIG. 18 is the fifth UI of the display screen in the sequence according to a second embodiment:

FIG. 19 is the sixth UI of the display screen in the sequence according to a second embodiment:

FIG. 20 is a UI of a display screen in a sequence of an HSA that self-selects users according to a third embodiment:

FIG. 21 is a UI of a display screen in a sequence of an HSA that self-selects users according to a fourth embodiment:

FIG. 22 is a UI of a display screen in a sequence of an HSA that self-selects users according to a fifth embodiment:

FIG. 23 is a UI of a display screen in a sequence of an HSA that self-selects users according to a sixth embodiment:

FIG. 24 is a UI of a display screen in a sequence of an HSA that self-selects users according to a seventh embodiment:

FIG. 25 is a UI of a display screen in a sequence of an HSA that self-selects users according to an eighth embodiment:

FIG. 26 is a UI of a display screen in a sequence of an HSA that self-selects users according to a ninth embodiment:

FIG. 27 is a UI of a display screen in a sequence of an HSA that self-selects users according to a tenth embodiment: and

FIG. 28 is a UI of a display screen in a sequence of an HSA that self-selects users according to an eleventh embodiment.

### DETAILED DESCRIPTION

As used throughout this application, the word “may” is used in a permissive sense (i.e., meaning having the potential to), rather than the mandatory sense (i.e., meaning must). The words “include,” “including,” and “includes” and the like mean including, but not limited to. As used herein, the singular form of “a,” “an,” and “the” include plural references unless the context clearly dictates otherwise. As employed herein, the term “number” shall mean one or an integer greater than one (i.e., a plurality).

As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other.

Unless specifically stated otherwise, as apparent from the discussion, it is appreciated that throughout this specification discussions utilizing terms such as “processing,” “computing,” “calculating,” “determining,” or the like refer to actions or processes of a specific apparatus, such as a special purpose computer or a similar special purpose electronic processing/computing device.

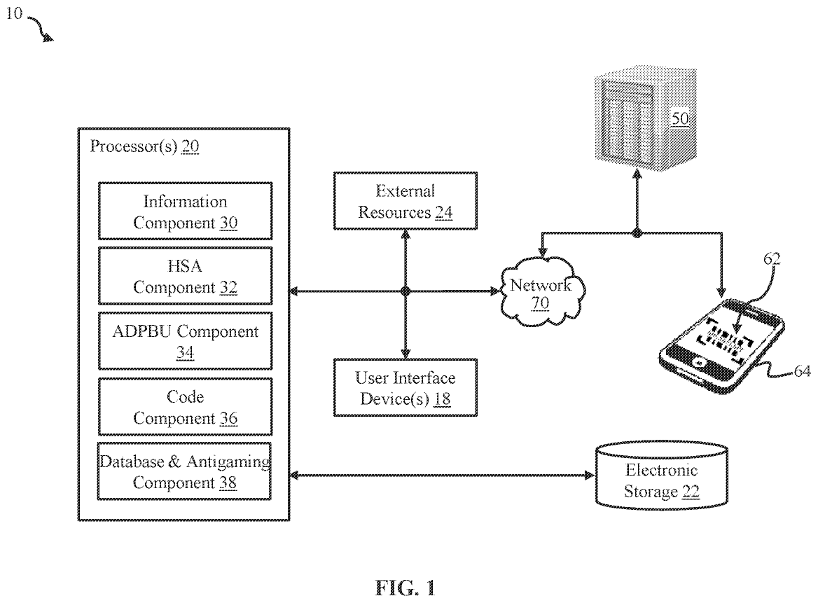

FIGS. 1, 5, 6A, and 6B illustrate system 10 configured to implement a medical algorithm that can evaluate a user's eligibility for safe use. For example, the health survey assessment (HSA) or initial questionnaire may be a digital tool that determines whether OTC drug 45 is suitable for consumer 40 based on their self-report to questions included in the HSA: the HSA may only provide purchase code 62 for resultant scores satisfying an eligibility criterion. The HSA is a selection tool (e.g., which may be hosted at a website or integrated into a web application (app)) that determines said suitability by sequentially asking questions and receiving confirmations, effectively helping a person self-select use of the medicine for a specific condition. The HSA may be built from the DFL or other labeling, and it may ensure safety for the consumer as they inform system 10 of attributes of their health.

The HSA may comprise a medical grade test for assessing safety of a user using nonprescription drug 45. For example, a health risk may be due to currently taking heart-related, nitrate-based medication and/or alpha blockers as part of blood pressure medicine. In this example, safety of user 40 will not be compromised, as consumers of Nitrate medications (i.e., those who self-report as “Yes” or “I don't know” to the do not use (DNU) warnings including such medicine) will be excluded from having access to drug 45 (e.g., Cialis) over-the-counter (OTC). The test of the HSA may be medical grade due at least to its questions and confirmations being classified and approved by a national regulatory body, such as the FDA.

In some embodiments, the HSA may implement assessment as to whether drug 45 may be used by the user safely and effectively, e.g., where the drug facts labeling (DFL) alone is not sufficient to ensure said safe and effective use of the drug product. For example, the HSA may comprise (1) development of labeling in addition to the DFL and (2) implementation of additional conditions so that the user appropriately self-selects and uses a drug product. Appropriateness and specific details of either of these approaches may depend on circumstances that apply to the particular drug product.

In some embodiments, HSA component 32 may implement qualifying condition(s) (e.g., algorithmically via the HSA to generate or unlock a code), and code component 36 may implement verification condition(s) (e.g., obtaining the code to enable a purchase). For example, a combination or sequence of UIs (e.g., screens 71, 72, 74, 75, 76, and/or 78) may perform algorithmic scoring to arrive at a qualification of user 40 for the purchase. Code component 36 may require a determined score to satisfy a criterion before generating or unlocking the code. For example, a condition may be reported by consumer 40 via an app or webpage, and a resultant score may cause this user to be denied or withheld access 110, restricted access, or given access 118 to purchase OTC drug 45.

Drug product 45 may be dispensed (e.g., via OTC at kiosk 50, OTC at a shipment to a home, or other suitable means). The dispensing means may be a medical device platform.

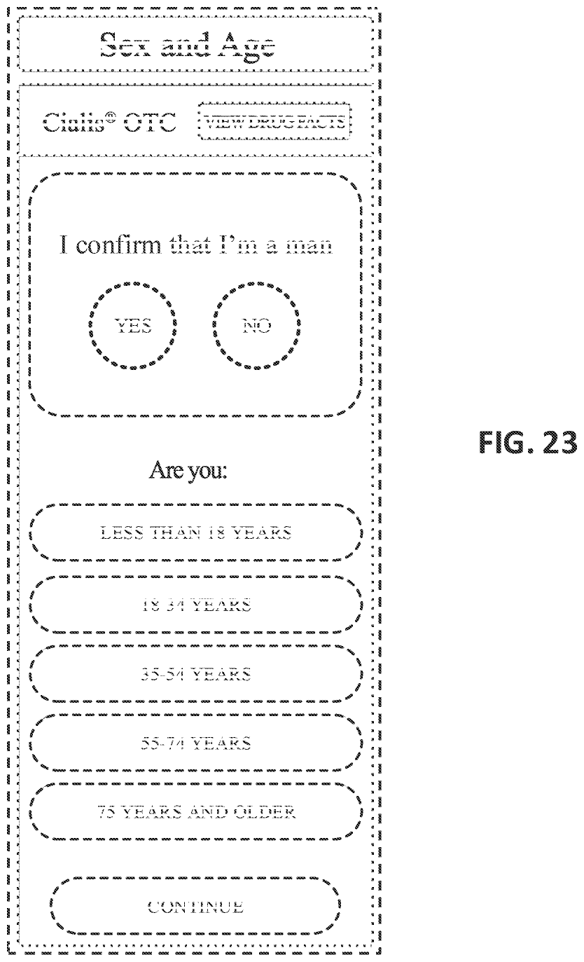

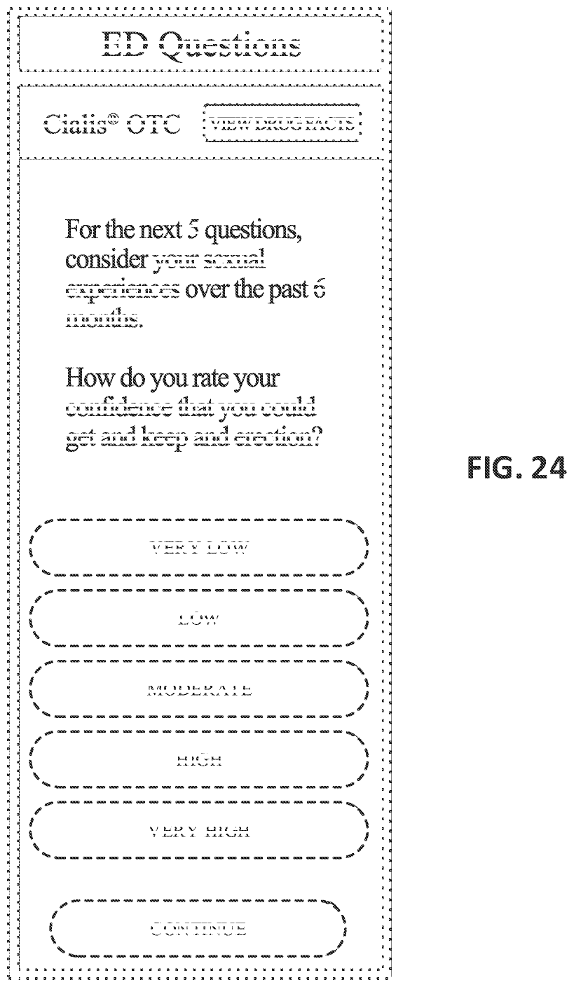

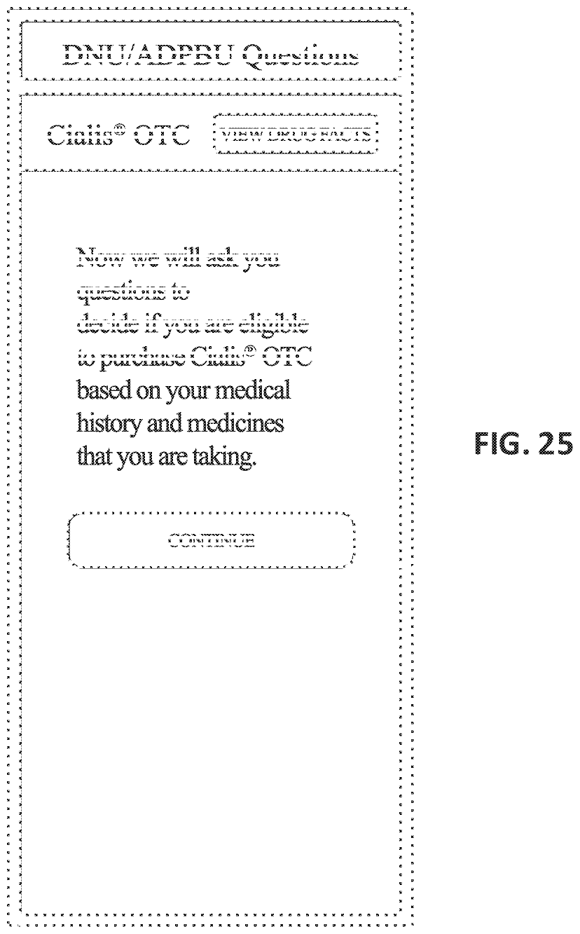

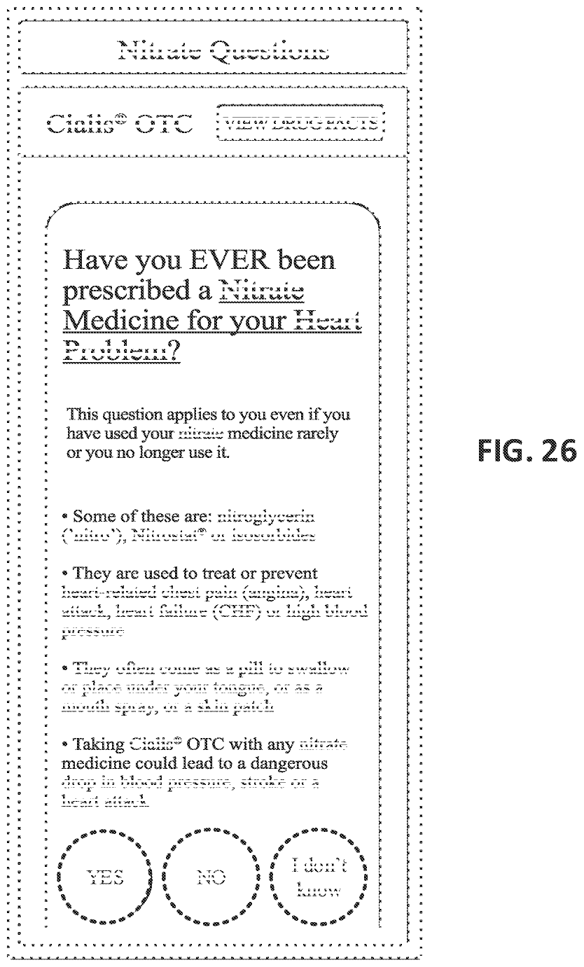

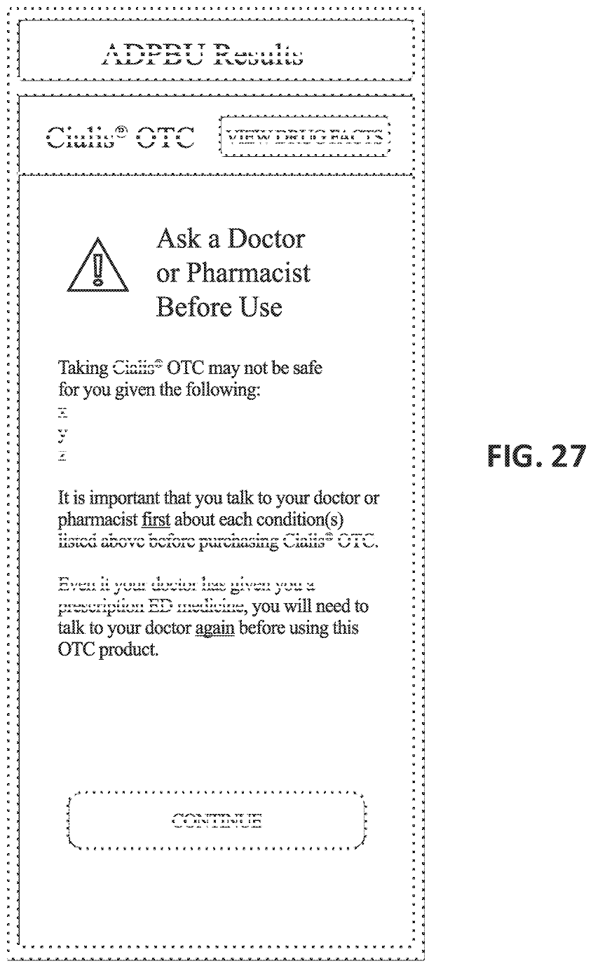

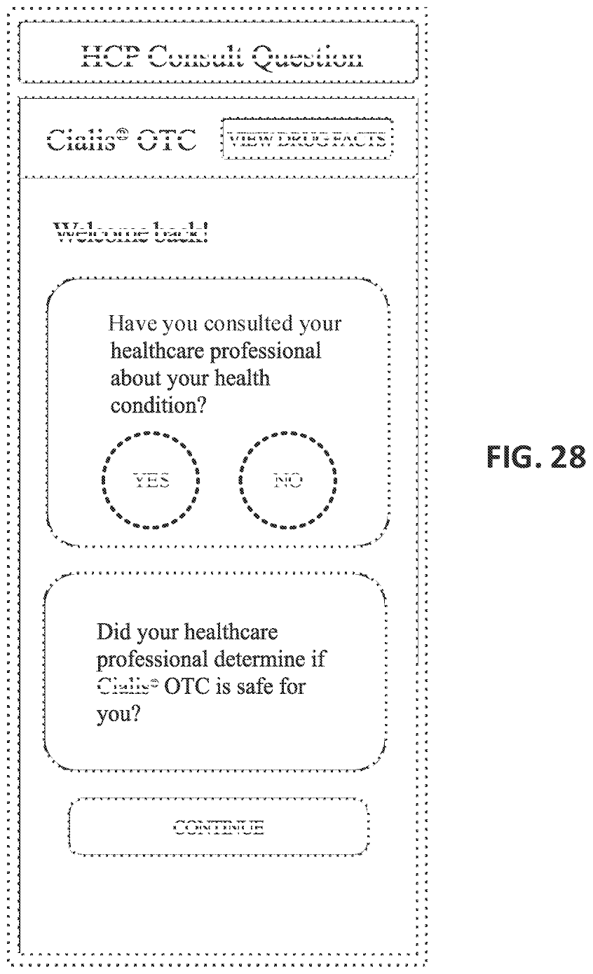

As shown in the example of FIGS. 6A-6B, kiosk 50 may dispense nonprescription drug product 45, which may comprise a set of pills, in a container, or a set of blisters, in a package, each blister in the package comprising a set of pills. The package or container may further comprise a consumer information leaflet and/or a sleeve for transporting a single blister.

In some embodiments, system 10 may dispense drug 45 in a safe, OTC, and effective way. For example, the drug may be suggested or recommended for use under conditions, e.g., which are properly labeled, in terms of content and formatting. This or other related labeling may be in addition to the DFL and include: information leaflets or other documents contained inside a carton or container housing drug 45: text or images on a video display, including interactive displays for consumers to review: information displayed on websites: and/or statements or questions in a mobile application.

Drug product 45 may treat chronic conditions or other conditions for which the limitations of the DFL present challenges for adequate communication of information needed for safe and effective use, e.g., without the supervision of the HCP. For example, drug 45 may be a contraindicated condition or medication, such as Cialis (Tadalafil) for erectile dysfunction (ED) or Mevacor (Lovastatin) for treating high blood cholesterol and reducing the risk of cardiovascular disease. The herein-disclosed approach enables a way to deal with one's health condition quickly and discreetly.

Nonprescription drug products 45 dispensed via a sequence of UIs improve health by increasing the types of drug products consumers can access and use that would otherwise only be available by prescription and by a pharmacist.

In some examples, the dispensation may be without the supervision of an HCP. In some embodiments, the HSA of user interface (UI) devices 18 improves existing technology by confirming whether the user applies information in the drug product's labeling to their personal medical situations and makes correct decisions to use or not use drug product 45. For example, code component 36 may output a code used in interaction with a purchasing component such that drug product 45 is dispensed (e.g., for safe, effective, and nonprescription use, when the DFL alone is not sufficient for this purpose). In this or another example, system 10) may implement additional conditions necessary for said safe and effective use. Examples of additional conditions for safe and effective use, particularly with regard to appropriate self-selection and actual use, may comprise: prior to purchase, the consumer is required to respond to a set of questions on a self-selection test performed at user equipment (UE) 64, and the outcome of the self-selection test affirmatively indicates that the consumer is an appropriate candidate to use the nonprescription drug product; and prior to purchase, the consumer is required to view and affirm that they viewed text or images in a video that describes how to use appropriately the nonprescription drug product.

In some embodiments, database component 38 may provide opportunities for consumer relationship marketing (CRM) based on data collected, e.g., to determine how processor 20 engages with consumers 40 via emails, consumer care, content delivery, and/or other messaging. In these or other embodiments, database component 38 may facilitate automation of resupply or repurchase of the drug product.

Consumers motivated to purchase drug 45 may simply deduce that a correct answer for all the questions regarding specific medications or medical conditions is no, to bypass these safeguards. In some embodiments, antigaming component 38 may implement one or more safeguards, to keep consumers from repeating the HSA until they successfully obtain purchase code 62. In these or other embodiments, antigaming component 38 may incorporate checks to look for duplicate users, i.e., those who attempt to enroll more than one time. In addition, the sequence or flow of the HSA content itself may minimize gaming (e.g., ED questions, grouping all DNU and then presenting the DNU stop screen, the ask a doctor before use (ADBU) summary, and restricting access).

In some embodiments, antigaming component 38 may implement a lockout period (e.g., 10-15 minutes or more) to discourage users from ignoring the advice to seek an HCP consultation and to lock someone out who might be trying to game the system by immediately clicking to the ask a doctor or pharmacist before use (ADPBU) follow-up screens.

Registration 102 may be required, in some embodiments. And in some UIs thereafter a determination to proceed or a do not use (DNU) may be determined responsive to answers and attestations (e.g., 9 or more warnings) provided by user 40. In an example, code component 36 may implement antigaming by not outputting the code without an HCP confirming safety for user 40 to use drug 45. In another example, user 40 may accurately self- report results of HCP consultation into system 10.

In some embodiments, HSA component 32 and/or ADPBU component 34 may communicate UIs for attestations, such as please do not falsify any information as this can be injurious to your health.

In some embodiments, antigaming component 38 may determine an exceeding of a recommended or allowable dosing and preclude code component 36 from outputting code 62 (e.g., on a screen of UE 64 to kiosk 50 or via other means triggering a shipment). For example, back-end records may be kept to authorize proper dosing (e.g., which may be different for groupings of users 40). In this example, the number of pills that a man can purchase may be restricted by components 36 and 38. These components may interact with or implement a purchasing and tracking of each purchase, e.g., where a certain amount is authorized in a time period. An attempt to purchase over the authorized quantity may not be allowed, code 62 not being made available to user 40.

In some embodiments, HSA component 32 may implement antigaming as part of the HSA by (i) not having a back button and (ii) disabling the browser's ability to go to the previous screen to prevent users from changing their response. In some embodiments, antigaming component 38 may incorporate Internet protocol (IP) address tracking, as an additional safeguard against inappropriate use, by ensuring no users 40 can retake the HSA. And component 38 may guard against spam, precluding robots from going through the HSA, learning the algorithm, and generating transferable approval codes.

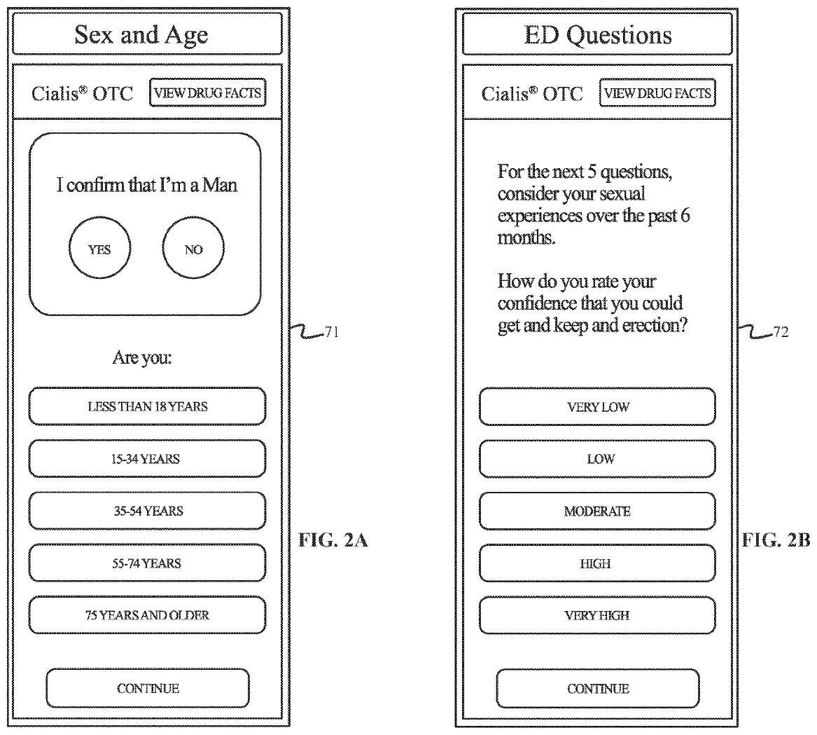

As shown in FIGS. 2A-4B, UIs 71, 72, 74, 75, 76, and 78 may be presented with continued offering of the electronic DFL (e.g., at the top right corner by a clickable button titled, “View Drug Facts”), for easy access to a familiar format for consumers.

In some embodiments, HSA component 32 may implement ease via visual cues, such as by keeping buttons inactive until something is selected and keeping the “Continue” button inactive until all questions have been answered on the screen: answering each question may be required to proceed to a next screen. In these or other embodiments, HSA component 32 may group related questions. For example, all of the drug-drug interaction ADPBU questions may be together via sequentially adjacent UIs so that consumers may answer all questions related to their medications in close time-proximity or real-time.

In some embodiments, HSA component 32 may also cause display of screens having helper text, with the exception of a few question screens (e.g., if you get dizzy or feel faint, take any blood pressure medicine, stomach ulcers, bleeding disorders, sickle cell anemia, leukemia or multiple myeloma) as the conditions are well understood: this may be performed rather than confusing the user with medical terminology. In an implementation, HSA component 32 may have a “click for more information” feature that conceals the helper text so that, when a consumer clicks on it, the helper text may appear. Further, the HSA may be made available across different devices, regardless of the size of the device and may ensure that the content is available through screen-readers or in key board-only modes. While the over screen presentation layer may vary (i.e., a mobile phone screen design will be slightly different from a tablet or desktop computer), the functionality of the application may not differ across devices. One implementation may comprise modules that automatically speak (e.g., robotic) or otherwise interact to the user.

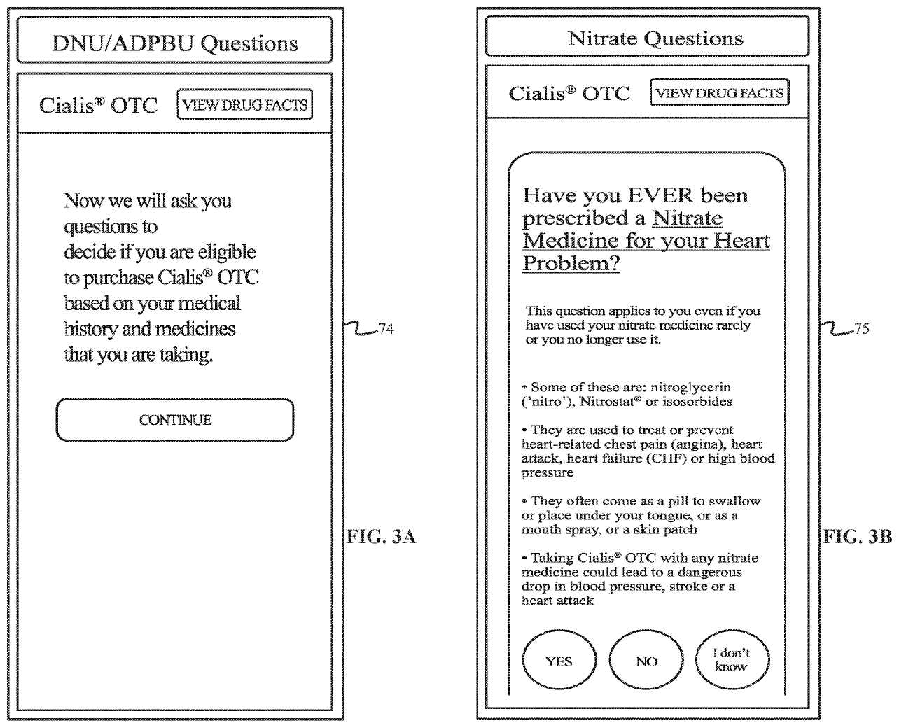

FIG. 2A depicts exemplary UI 71, wherein initial questions on user 40's personal attributes are asked, such as relating to sex and age. For example, user 40 may attest that they are male sex, and that their age is 18 years or older. Once user 40 has qualified that, they may be authorized to proceed to the medical or health condition question, such as the one asked in UI 72 of FIG. 2B. Once HSA component 32 determines that user 40 has the medical condition (e.g., ED), then they may go to the next set of questions which are the warning questions. That is, once the consumer has answered a set of questions in one UI, the consumer is able to move on to the next set of questions (e.g., from FIG. 2A to FIG. 2B to FIG. 3A, etc.). The conditional question of FIG. 2B may, in some implementations, be answered as a result of consultation with an HCP.

Consumer 40 may be asked to affirm that they alone will be using the product. And a UI may be displayed warning the user that it is important to accurately report their health condition, as failure to do so can lead to serious health consequences. By clicking “Continue,” they may attest to understanding this warning.

FIG. 3A depicts an example compliance question, i.e., a do not use (DNU) and ADPBU question. For example, a user may self-report “No” for the DNU condition. But if a DFL warning applies (e.g., consumer self-reports “Yes” or “I don't know” to a DNU condition), consumer 40 may not receive purchase code 62, and the HSA path associated with the consumer's account may be captured and linked by antigaming component 38 so that user 40 may not be able to repeat the HSA and game the system to fraudulently obtain code 62.

Any consumer 40 who has a DNU warning may be denied OTC access to drug 45 and may have to exit the HSA. As an example, men who have self-reported the use of a nitrate medicine or have ever been prescribed a nitrate medicine will be denied access to Cialis OTC with a “Stop” screen, exiting the HSA: they may not receive purchase code 62. As such, HSA component 32 may deny access to drug 45 if the subject reports DNU conditions. The HSA may restrict first access to the drug, if the subject reports ADBU or ADPBU conditions. Access to these users may only be provided if the subject then reports back into the HSA that their doctor has determined it is safe to use drug 45.

FIG. 3B depicts UI 75 with an example question as to related health risks, e.g., with respect to consumption of a nitrate medicine. But this example may not be limiting, as upwards of 22 warnings in the DNU and ADPBU sections of the DFL may be included in the HSA. The HSA is thus a full selection tool including all warnings listed in the DNU, ADBU, and ADPBU sections of the DFL. As such, the HSA may implement a sequence of UIs as condition of purchase for user 40, potentially without involvement of any other human. The UIs improve relevant technology, e.g., by keeping users 40 at the center of their decision-making and to facilitate navigation. For example, a UI may be displayed on a screen to advise consumers to read the DFL and so that they do not use Cialis OTC with any other ED medicine.

With this nitrate question of the example of FIG. 3B, the consumer may self-report either “Yes” or “I don't know,” that they have ever been prescribed a nitrate medicine. Then, they may get a DNU screen, denying them access to the medicine, if a user confirms to having a DNU condition (e.g., taking nitrate medicine); users 40 being displayed such a screen may not go any further in the HSA. Similarly, the HSA may restrict first-time users from purchasing drug 45 who respond “Yes” or “I don't know” to the ADBU/ADPBU warning conditions.

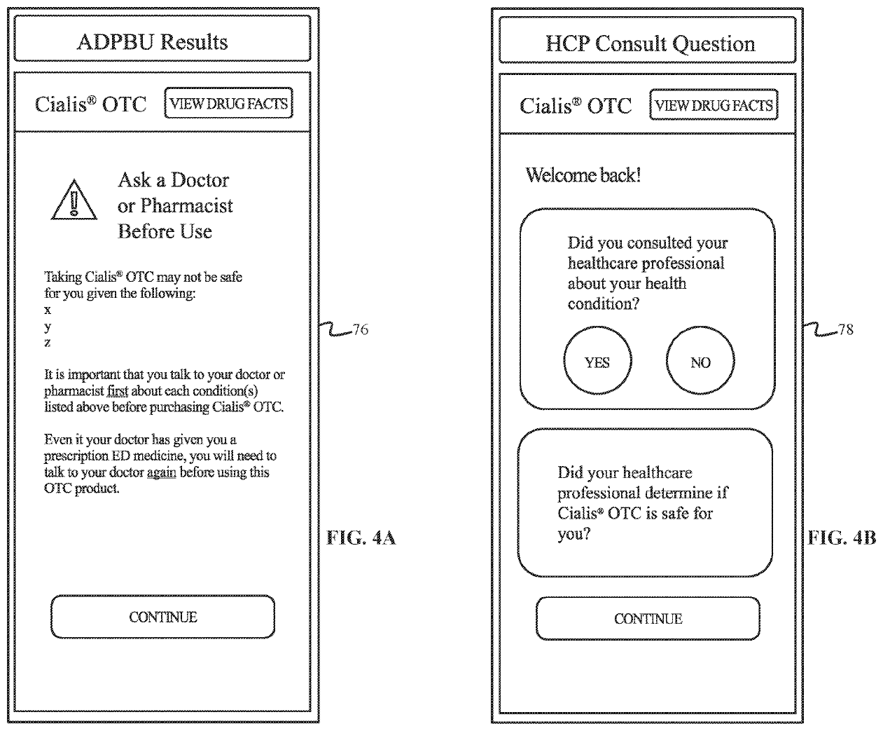

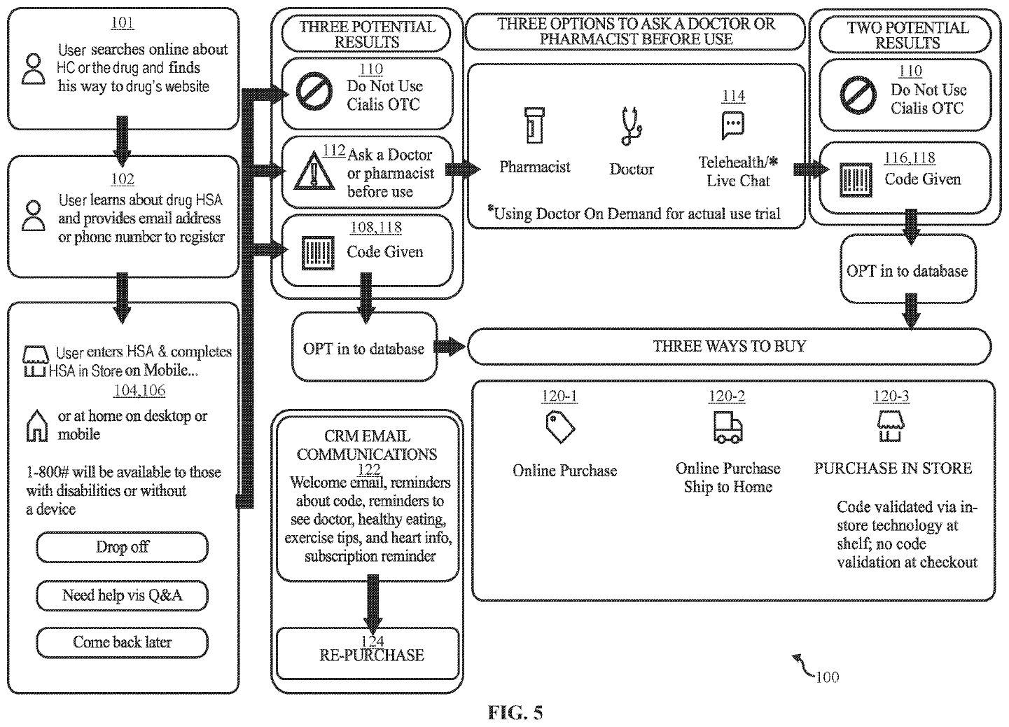

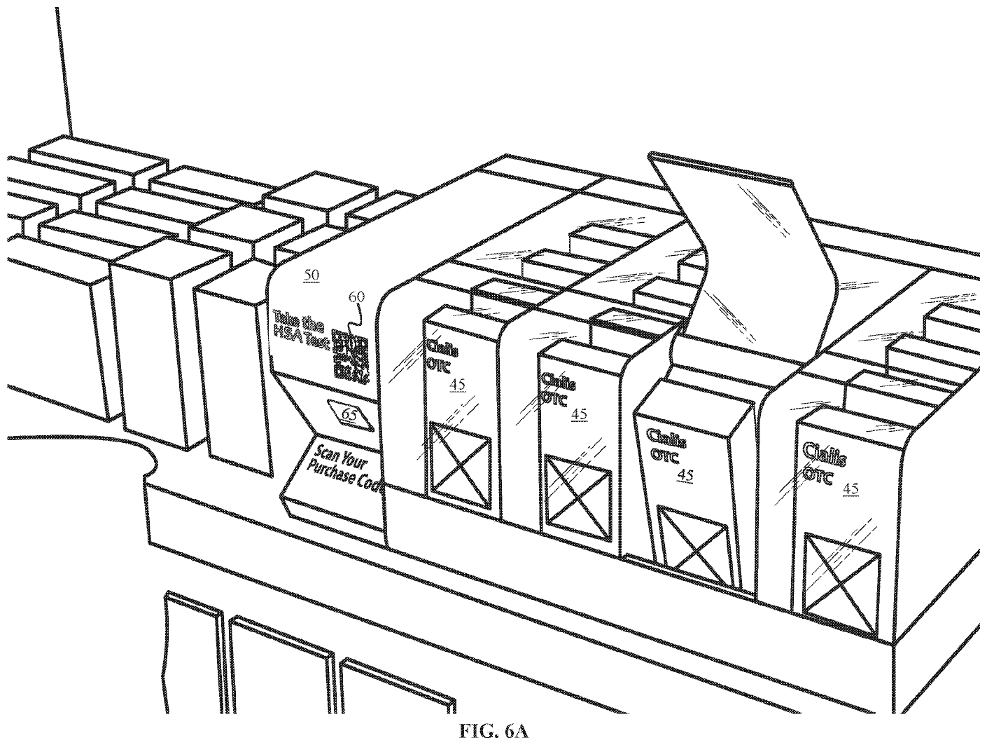

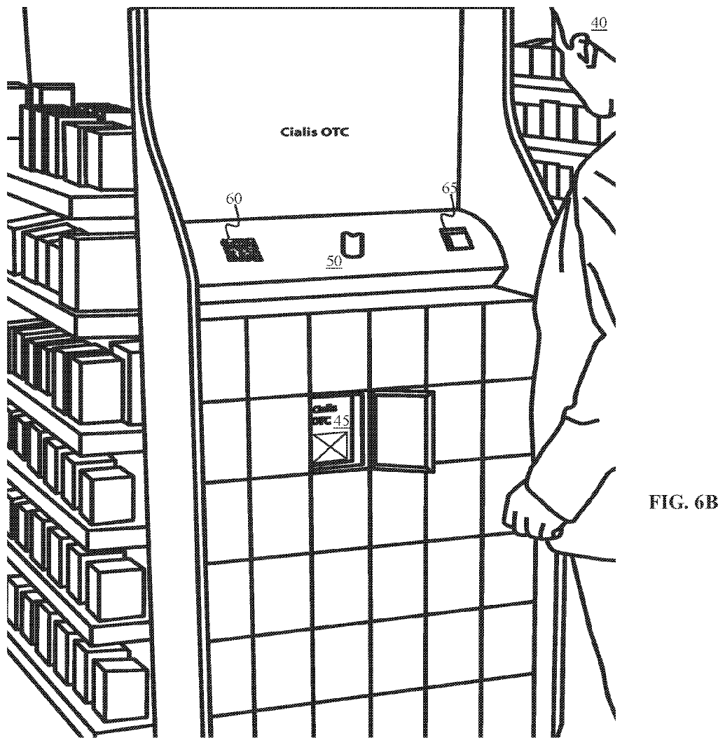

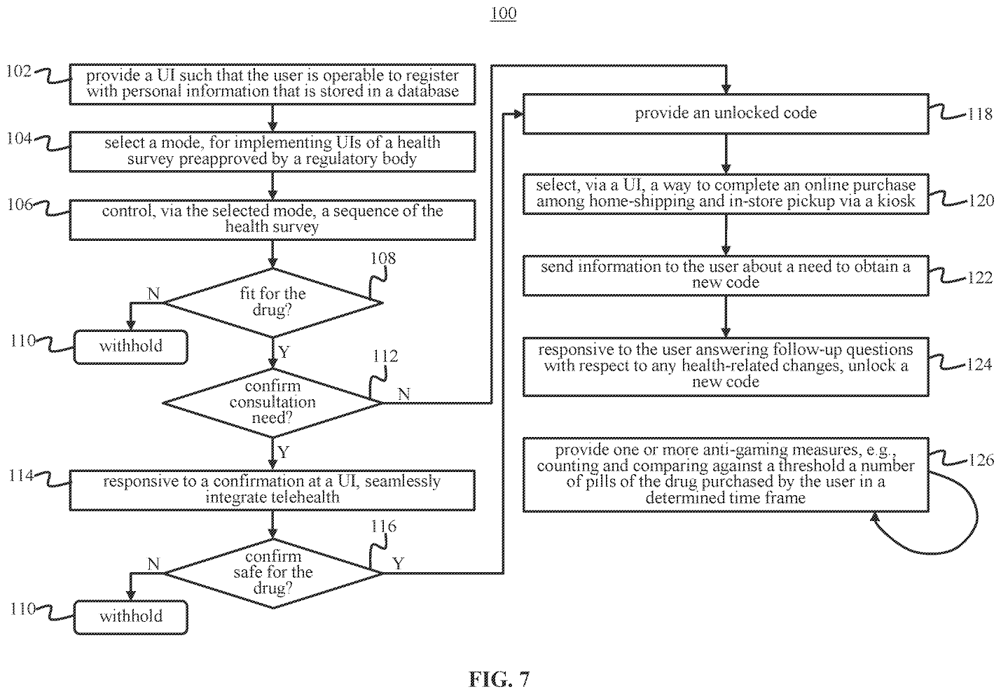

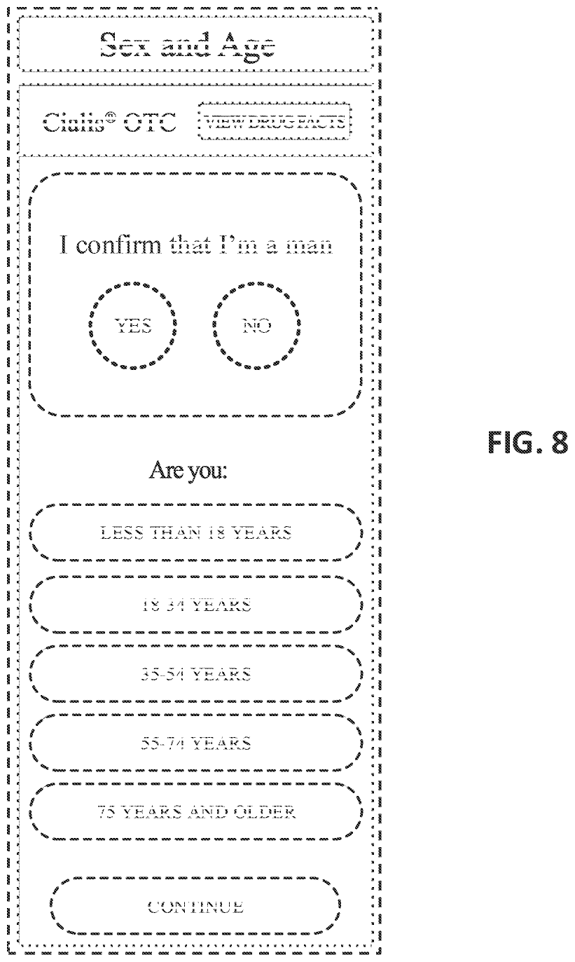

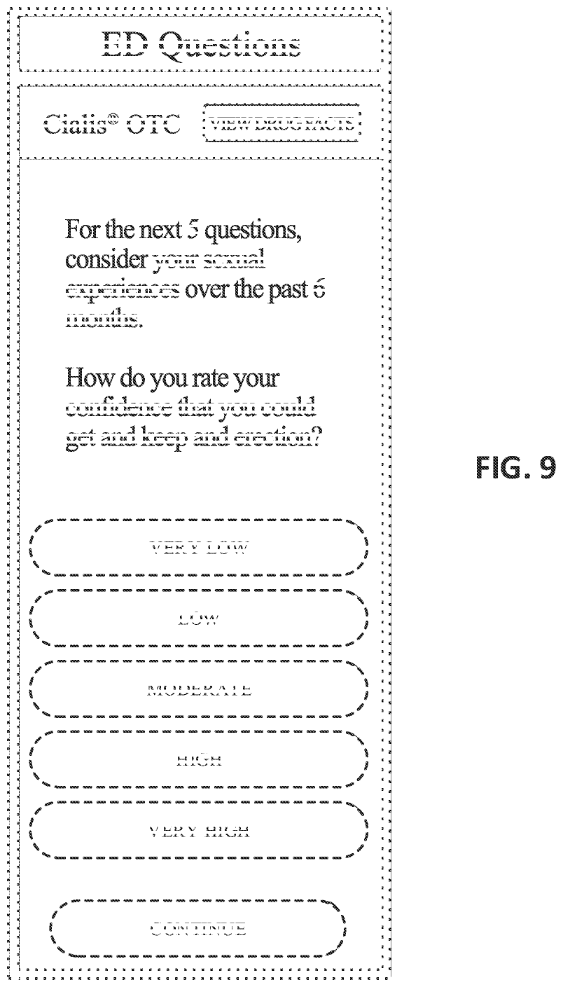

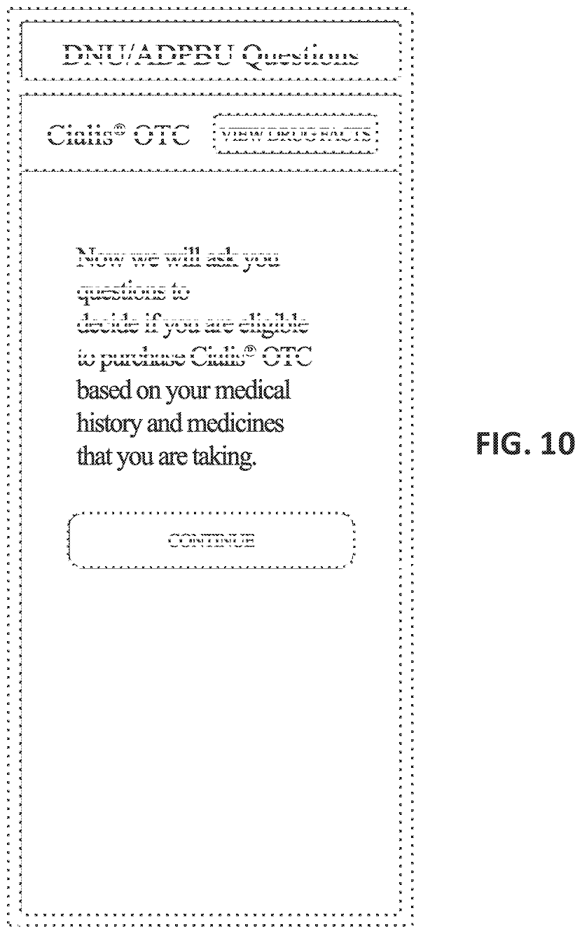

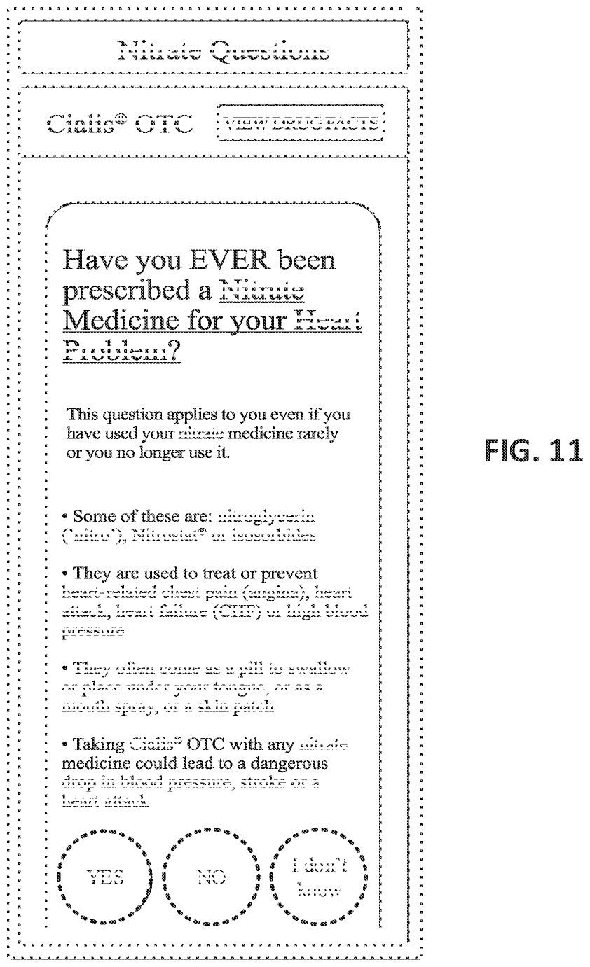

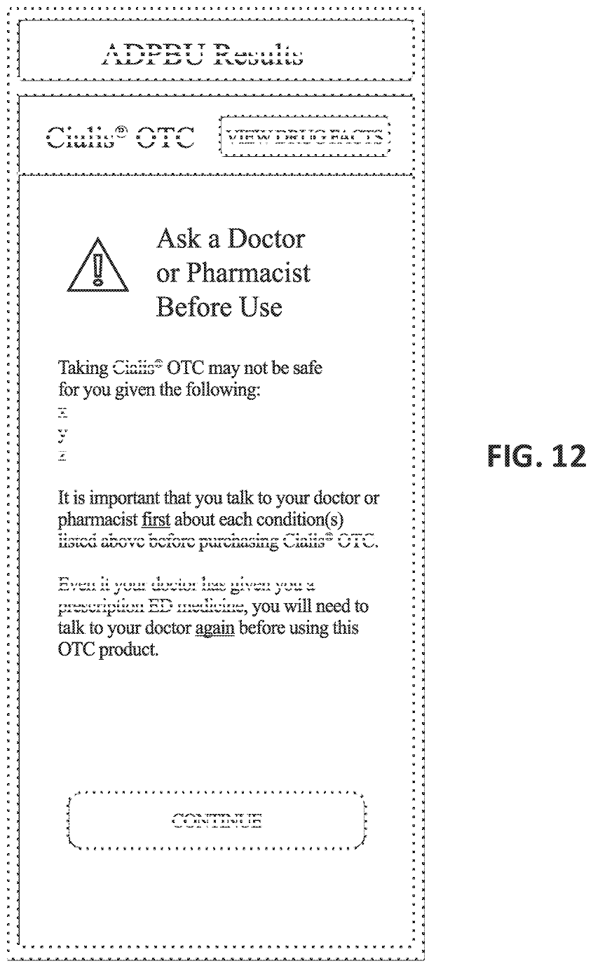

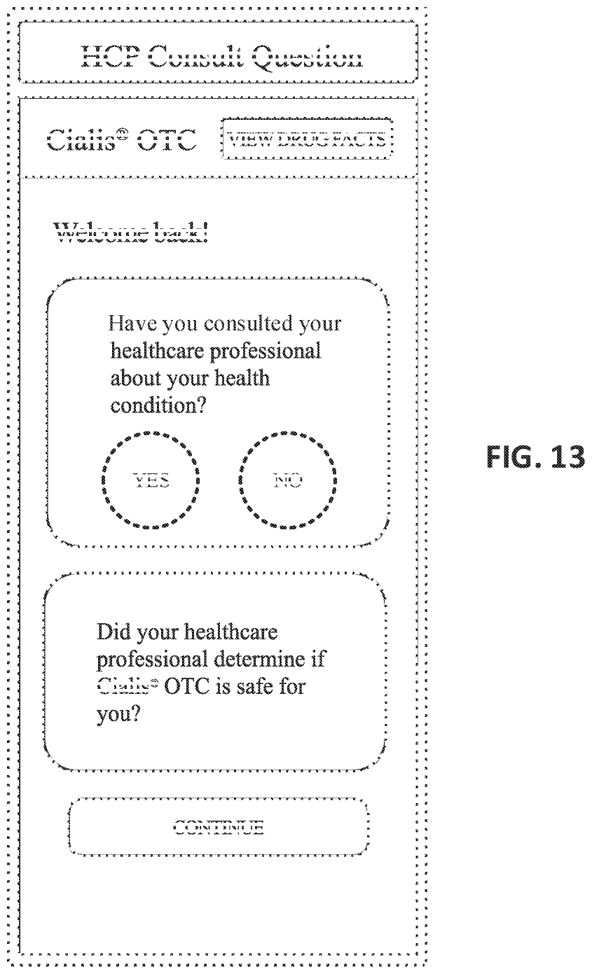

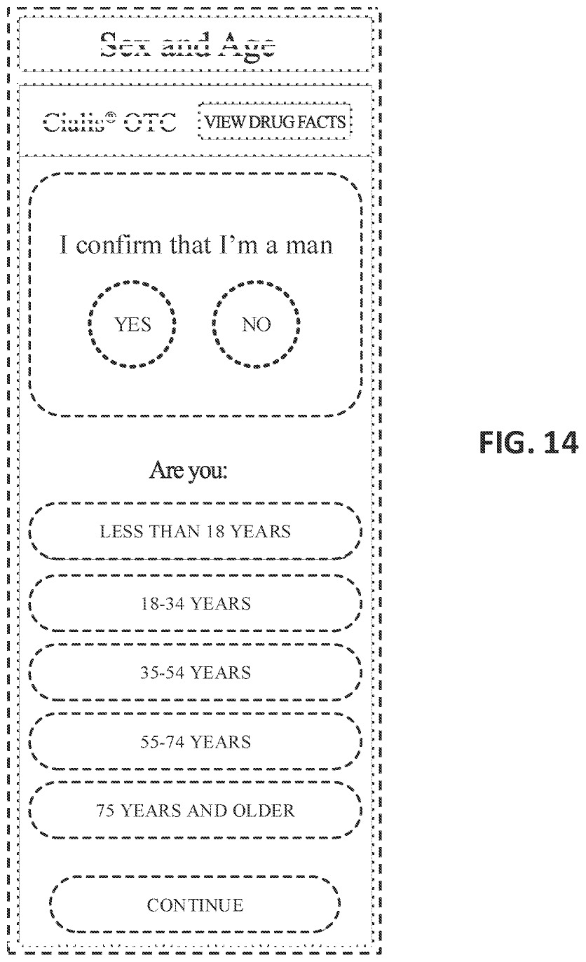

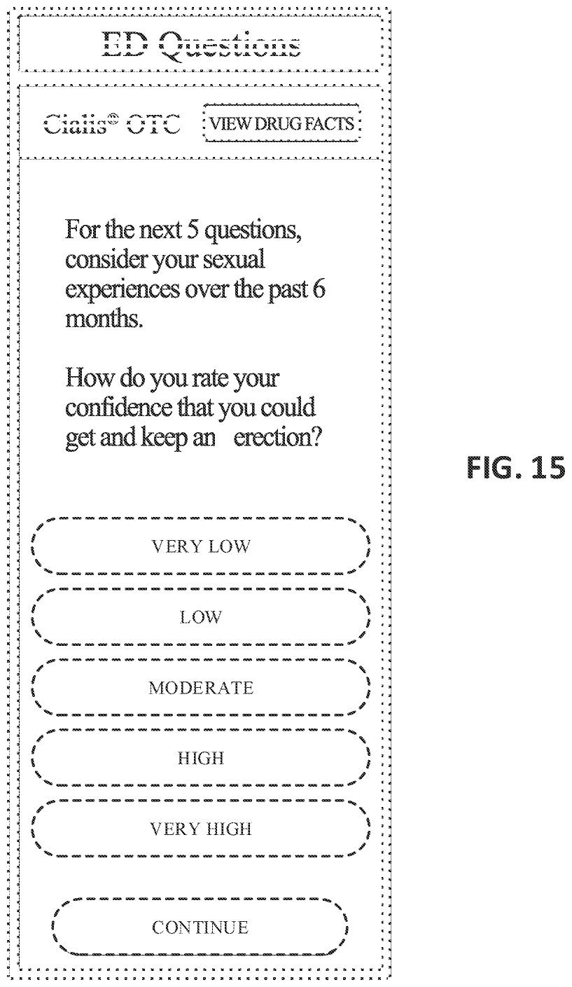

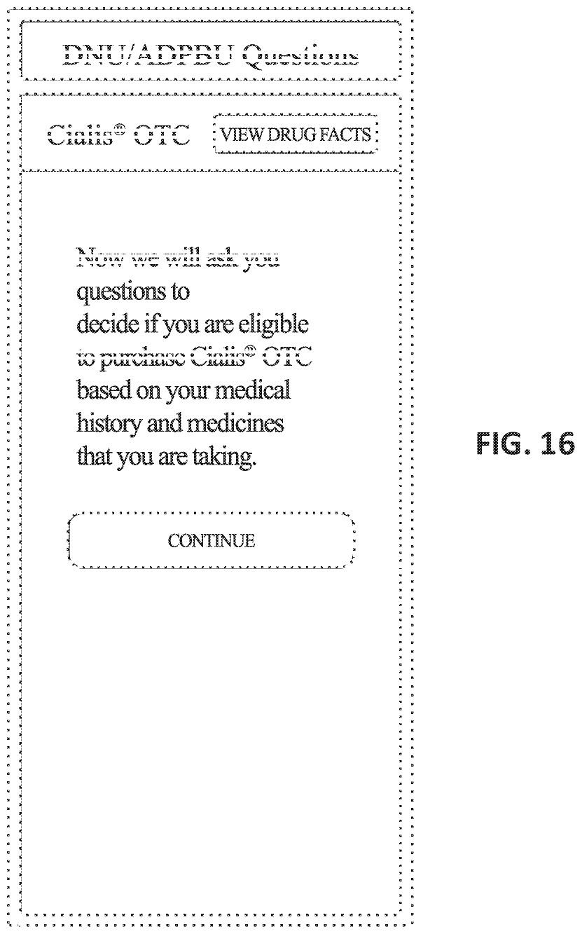

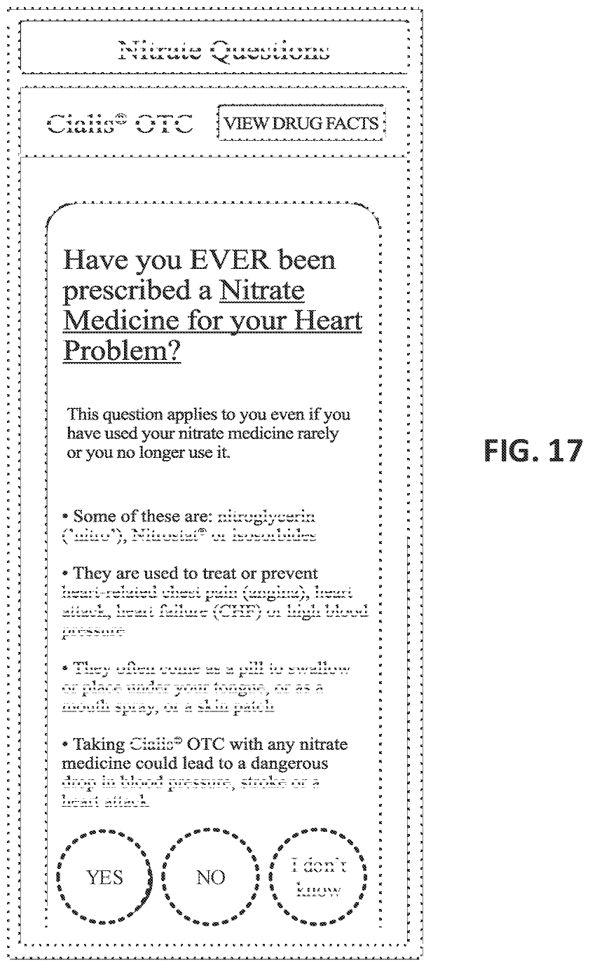

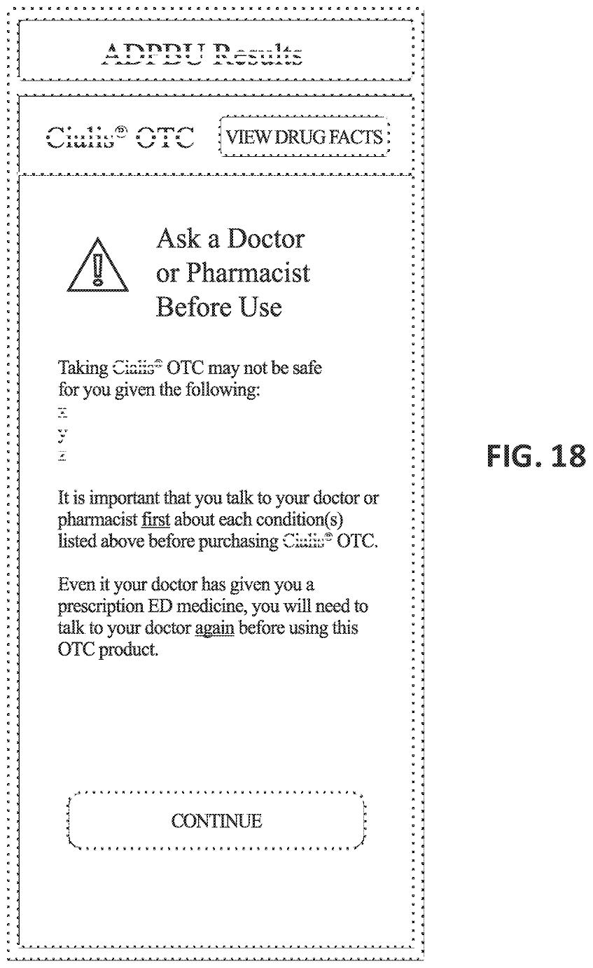

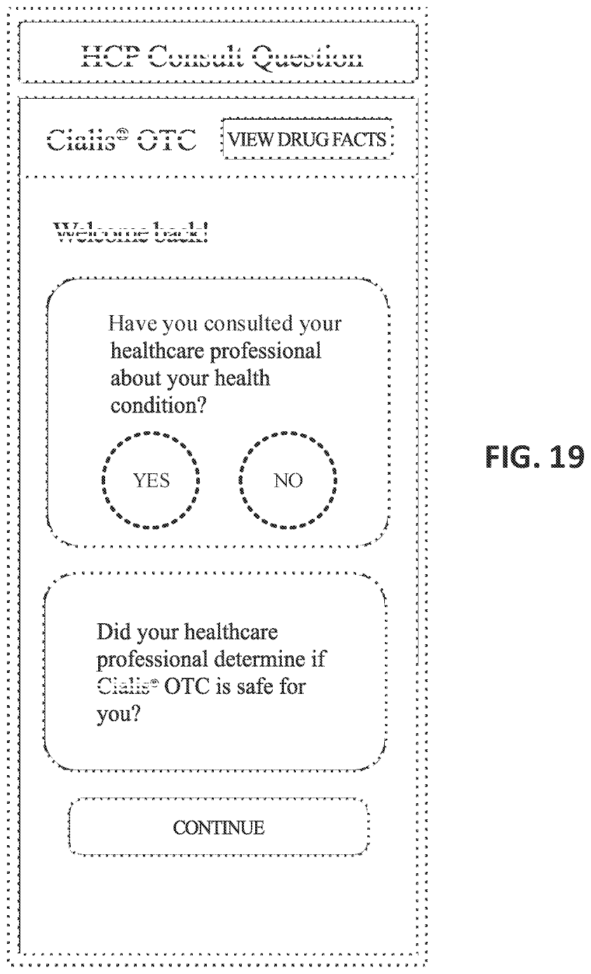

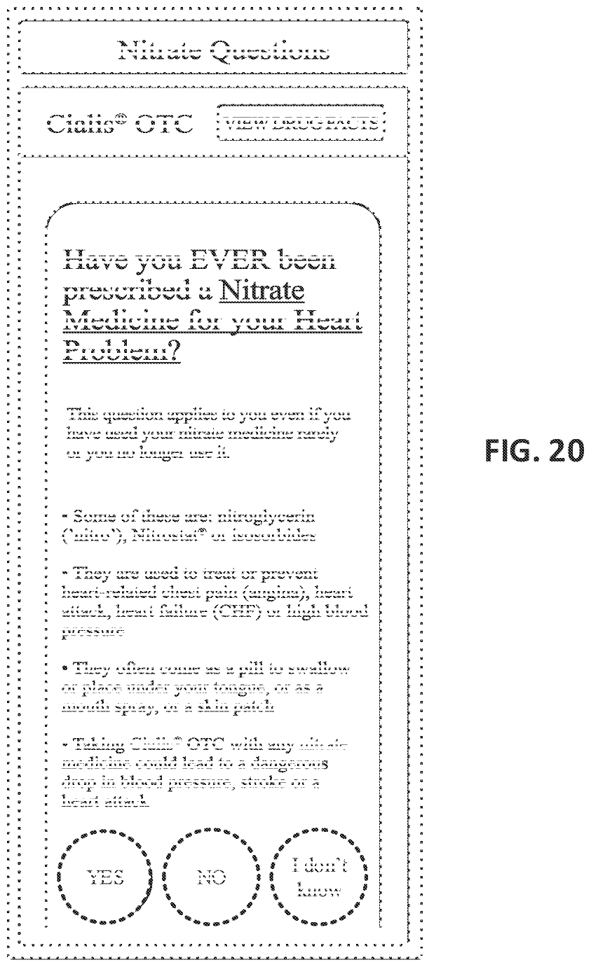

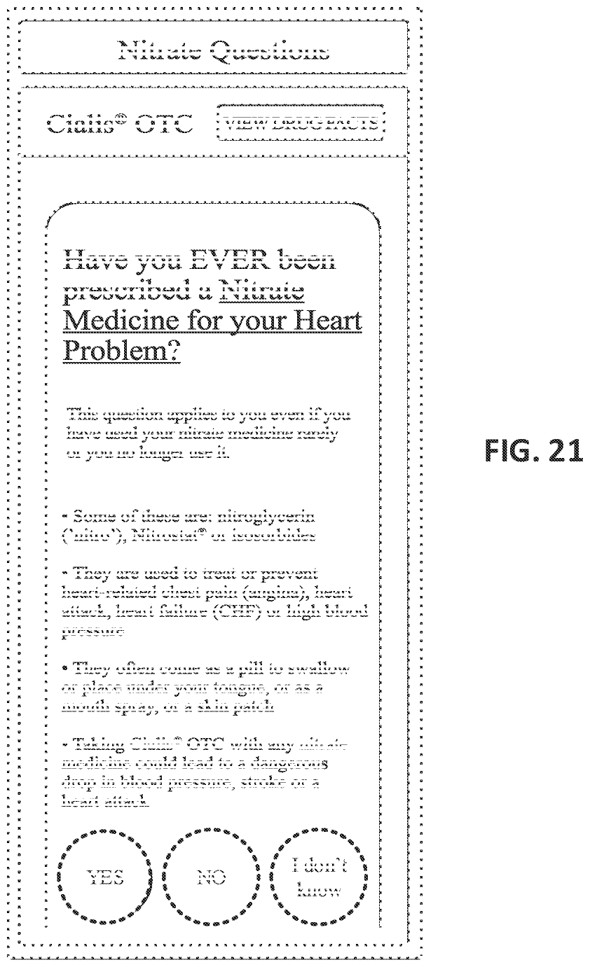

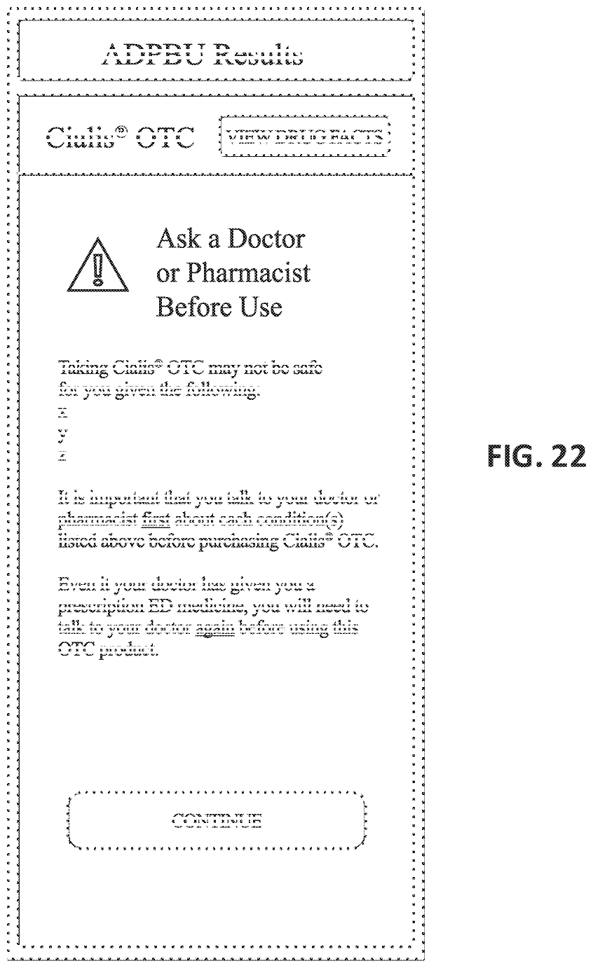

FIG. 4A depicts an example of self-reporting a “Yes,” for doctor confirmation of safe use if an ADBU/ADPBU condition applies. But if user 40 is alerted with need to ask a doctor before use, there can be more than one way to do so. For example, the user may have three options to be able to go and get a consultation, including consulting a pharmacist in store, a doctor at their clinic, or they may have a telehealth service that ADPBU component may provide as an opportunity to interact with a doctor either by phone call or by live chat. Out of that conversation, they may either receive a DNU, a do not proceed, or they may get code 62 that they need to purchase. An opportunity to opt into database 22 for messaging and marketing communications may be provided.

The remaining consumers 40 may all go to the ADPBU question, including a series of questions. Once the user self-reports that they have a heart problem (e.g., which may be one of the ADBU condition), then they will get UI 76. As a result, user 40 may be automatically taken out of the HSA. The user may be given 30 days to go consult with their doctor, and once the HCP has indicated yes, they may come back and report that consultation that the HCP has given an okay, as depicted in UI 78 of FIG. 4B. FIG. 4B is thus an example of conditional confirmation, i.e., responsive to a previously determined need to initially consult the HCP.

At both FIGS. 2A and 4B, user 40 may be informed that by clicking “Continue” that they confirm understanding that falsifying information may lead to serious health consequences, to remind consumers that it is important for them to be transparent about their health condition when responding to the survey.

FIGS. 6A and 6B each depict exemplary kiosk technology inside of a retail store that may secure drug 45 for OTC procurement, e.g., only allowing appropriate users 40 who have completed the HSA and are in possession of the approval code to obtain a drug therefrom. A completed registration and approval code may thus be required for each purchase. For example, kiosk 50 may comprise code reader 65 to read code 62 from UE 64. In another example, the user may electronically emit code 62 to code component 36.

In some embodiments, upon UE 64 obtaining and emitting (e.g., visually via the screen, as depicted in FIG. 1, or wirelessly via Bluetooth, near field communication (NFC), or other means) the unlocked or generated code, an external system connected via network 70 (or kiosk 50 alone) may check if the code is valid against a back-end database before unlocking a door at kiosk 50. In some embodiments, each of HSA code 60 and purchasing code 62 may be a quick response (QR), bar code, alphanumeric code, Aztec code, or another suitable code. In these or other embodiments, UE 64 may be directed to the HSA via other means (e.g., when purchasing online for home shipment such that code 60 is unnecessary), and/or UE 64 may become authorized to purchase drug 45 via other means (e.g., when code 62 is not displayed but rather is communicated wirelessly to kiosk 50 using an antenna).

Electronic storage 22 of FIG. 1 comprises electronic storage media that electronically stores information. The electronic storage media of electronic storage 22 may comprise system storage that is provided integrally (i.e., substantially non-removable) with system 10 and/or removable storage that is removably connectable to system 10 via, for example, a port (e.g., a USB port, a firewire port, etc.) or a drive (e.g., a disk drive, etc.). Electronic storage 22 may be (in whole or in part) a separate component within system 10, or electronic storage 22 may be provided (in whole or in part) integrally with one or more other components of system 10 (e.g., a UI device 18, processor 20, etc.). In some embodiments, electronic storage 22 may be located in a server together with processor 20, in a server that is part of external resources 24, in UI devices 18, and/or in other locations. Electronic storage 22 may comprise a memory controller and one or more of cloud-based storage, optically readable storage media (e.g., optical disks, etc.), magnetically readable storage media (e.g., magnetic tape, magnetic hard drive, floppy drive, etc.), electrical charge-based storage media (e.g., EPROM, RAM, etc.), solid-state storage media (e.g., flash drive, etc.), and/or other electronically readable storage media. Electronic storage 22 may store software algorithms, information obtained and/or determined by processor 20, information received via UI devices 18 and/or other external computing systems, information received from external resources 24, and/or other information that enables system 10 to function as described herein.

External resources 24 may include sources of information (e.g., databases, websites, etc.), external entities participating with system 10, one or more servers outside of system 10, a network, electronic storage, equipment related to Wi-Fi technology, equipment related to Bluetooth® technology, data entry devices, a power supply (e.g., battery powered or line-power connected, such as directly to 110 volts AC or indirectly via AC/DC conversion), a transmit/receive element (e.g., an antenna configured to transmit and/or receive wireless signals), a network interface controller (NIC), a display controller, a graphics processing unit (GPU), and/or other resources. In some implementations, some or all of the functionality attributed herein to external resources 24 may be provided by other components or resources included in system 10. Processor 20, external resources 24, UI device 18, electronic storage 22, a network, and/or other components of system 10 may be configured to communicate with each other via wired and/or wireless connections, such as a network (e.g., a local area network (LAN), the Internet, a wide area network (WAN), a radio access network (RAN), a public switched telephone network (PSTN), etc.), cellular technology (e.g., GSM, UMTS, LTE, 5G, etc.), Wi-Fi technology, another wireless communications link (e.g., radio frequency (RF), microwave, infrared (IR), ultraviolet (UV), visible light, cm wave, mm wave, etc.), a base station, and/or other resources.

UI device(s) 18 of system 10 may be configured to provide an interface between one or more users and system 10. UI devices 18 are configured to provide information to and/or receive information from the one or more users. UI devices 18 include a UI and/or other components. The UI may be and/or include a graphical UI configured to present views and/or fields configured to receive entry and/or selection with respect to particular functionality of system 10, and/or provide and/or receive other information. In some embodiments, the UI of UI devices 18 may include a plurality of separate interfaces associated with processors 20 and/or other components of system 10. Examples of interface devices suitable for inclusion in UI device 18 include a touch screen, a keypad, touch sensitive and/or physical buttons, switches, a keyboard, knobs, levers, a display, speakers, a microphone, an indicator light, an audible alarm, a printer, and/or other interface devices. The present disclosure also contemplates that UI devices 18 include a removable storage interface. In this example, information may be loaded into UI devices 18 from removable storage (e.g., a smart card, a flash drive, a removable disk) that enables users to customize the implementation of UI devices 18.

In some embodiments, UI devices 18 are configured to provide a UI, processing capabilities, databases, and/or electronic storage to system 10. As such, UI devices 18 may include processors 20, electronic storage 22, external resources 24, and/or other components of system 10. In some embodiments, UI devices 18 are connected to a network (e.g., the Internet). In some embodiments, UI devices 18 do not include processor 20, electronic storage 22, external resources 24, and/or other components of system 10, but instead communicate with these components via dedicated lines, a bus, a switch, network, or other communication means. The communication may be wireless or wired. In some embodiments, UI devices 18 are laptops, desktop computers, smartphones, tablet computers, and/or other UI devices.

Data and content may be exchanged between the various components of the system 10 through a communication interface and communication paths using any one of a number of communications protocols. In one example, data may be exchanged employing a protocol used for communicating data across a packet-switched internetwork using, for example, the Internet Protocol Suite, also referred to as TCP/IP. The data and content may be delivered using datagrams (or packets) from the source host to the destination host solely based on their addresses. For this purpose the IP defines addressing methods and structures for datagram encapsulation. Of course other protocols also may be used. Examples of an Internet protocol include IP version 4 (IPv4) and IP version 6 (IPv6).

In some embodiments, although processor 20 (and other components of system 10) is shown distinct from UE 64 in FIG. 1, one or more of functionality implemented therein may be implemented by a processor of UE 64. That is, one or more aspects of processor(s) 20 may be in another device or integrated with (e.g., in a same or separate housing) user device 64, which may be a consumer electronics device, a mobile phone, a smartphone, a personal data assistant, a digital tablet/pad computer, a wearable device (e.g., watch), augmented reality (AR) goggles, virtual reality (VR) goggles, a reflective display, a personal computer, a laptop computer, a notebook computer, a work station, a server, a high performance computer (HPC), a vehicle (e.g., embedded computer, such as in a dashboard or in front of a seated occupant of a car or plane), a game or entertainment system, a set-top-box, a monitor, a television (TV), a panel, or any other device. In some embodiments, processor 20 is configured to provide information processing capabilities in system 10. Processor 20 may comprise one or more of a digital processor, an analog processor, a digital circuit designed to process information, an analog circuit designed to process information, a state machine, and/or other mechanisms for electronically processing information. Although processor 20 is shown in FIG. 1 as a single entity, this is for illustrative purposes only. In some embodiments, processor 20 may comprise a plurality of processing units. These processing units may be physically located within the same device (e.g., a server), or processor 20 may represent processing functionality of a plurality of devices operating in coordination (e.g., one or more servers, UI devices 18, devices that are part of external resources 24, electronic storage 22, and/or other devices).

As shown in FIG. 1, processor 20 is configured via machine-readable instructions to execute one or more computer program components. The computer program components may comprise one or more of information component 30, HSA component 32, ADPBU component 34, code component 36, database and antigaming component 38, and/or other components. Processor 20 may be configured to execute components 30, 32, 34, 36, and/or 38 by: software; hardware; firmware; some combination of software, hardware, and/or firmware: and/or other mechanisms for configuring processing capabilities on processor 20.

It should be appreciated that although components 30, 32, 34, 36, and 38 are illustrated in FIG. 1 as being co-located within a single processing unit, in embodiments in which processor 20 comprises multiple processing units, one or more of components 30, 32, 34, 36, and/or 38 may be located remotely from the other components. For example, in some embodiments, each of processor components 30, 32, 34, 36, and 38 may comprise a separate and distinct set of processors. The description of the functionality provided by the different components 30, 32, 34, 36, and/or 38 described below is for illustrative purposes, and is not intended to be limiting, as any of components 30, 32, 34, 36, and/or 38 may provide more or less functionality than is described. For example, one or more of components 30, 32, 34, 36, and/or 38 may be eliminated, and some or all of its functionality may be provided by other components 30, 32, 34, 36, and/or 38. As another example, processor 20 may be configured to execute one or more additional components that may perform some or all of the functionality attributed below to one of components 30, 32, 34, 36, and/or 38.

FIGS. 5 and 7 illustrate method 100 for self-selecting provision of a drug, in accordance with one or more embodiments. Method 100 may be performed with a computer system comprising one or more computer processors and/or other components. The processors are configured by machine readable instructions to execute computer program components. The operations of method 100 presented below are intended to be illustrative. In some embodiments, method 100 may be accomplished with one or more additional operations not described, and/or without one or more of the operations discussed. Additionally, the order in which the operations of method 100 are illustrated in FIGS. 5 and 7 and described below is not intended to be limiting. In some embodiments, method 100 may be implemented in one or more processing devices (e.g., a digital processor, an analog processor, a digital circuit designed to process information, an analog circuit designed to process information, a state machine, and/or other mechanisms for electronically processing information). The processing devices may include one or more devices executing some or all of the operations of method 100 in response to instructions stored electronically on an electronic storage medium. The processing devices may include one or more devices configured through hardware, firmware, and/or software to be specifically designed for execution of one or more of the operations of method 100.

At operation 101 of method 100, user 40 may search online about a health condition (HC) (e.g., ED) or drug product 45 (e.g., Cialis) and navigate online to a website (e.g., Cialis.com). In an example, different uniform resource locators (URLs) may be used, such as one for the initial use of the HSA and different other ones for subsequent use of the HSA (e.g., for returning after talking to their doctor or pharmacist or for returning to purchase additional medicine 45 at a later date). In some embodiments, operation 101 is performed at a touchscreen of UE 64.

At operation 102 of method 100, a UI may be provided such that user 40 is operable to register with personally identifiable information (PII) that is stored in a database. For example, the user may learn about the HSA for drug 45 and obtain instructions to access the HSA through a web address with credentials to register, e.g., by providing a password and a legal name, date of birth, email address, mailing address, phone number, or a link to an existing account (e.g. Apple, Google, Facebook, etc.). As such, those interested in taking drug 45 OTC may be required to register an account to access the HSA. In some implementations, consumer 40's PII may be used in compliance with relevant applicable laws and regulations. In some embodiments, operation 102 is performed by a processor component the same as or similar to information component 30 (shown in FIG. 1 and described herein).

At operation 104 of method 100, a mode may be selected, for implementing UIs of a health survey preapproved by a regulatory body. As an example, user 40 may complete the HSA (e.g., online) using their own device 64 (e.g., laptop, tablet, mobile phone, as mentioned) by initially navigating to a particular webpage (e.g., www.cialis.com) or by opening the app, which may be implemented by HSA component 32. As such, the different modes may comprise at least one of an app-based questionnaire, web-based questionnaire, phone call with numerical prompts (e.g., for those with a disability), a short message service (SMS) chat bot, and other suitable means. In some embodiments, operation 104 is performed at UE 64.

At operation 106 of method 100, a sequence of the health survey may be controlled, via the selected mode. As an example, a series of questions and attestations may be progressively displayed in UIs at UE 64. In this example, display of screens 72, 74, 75, 76, and/or 78 may be determined based on the selected answer of previous question(s): that is, an initial screen (e.g., 71) may be predetermined, but all subsequent screens may be conditionally displayed. In some embodiments, operation 106 is performed by a processor component the same as or similar to HSA component 32 (shown in FIG. 1 and described herein).

At operation 108 of method 100, whether user 40 is fit for the drug may be determined. The DFL may comprise one or more labels similar to those of OTC products. Accordingly, the examples of FIG. 4A may implement aspects of a DFL with distinct options, such as DNU, ADBU, and ADPBU sections, each having different conditions and medications. For example, product 45 may be deemed not suitable for user 40, this DNU condition being based on the user taking a certain medication or certain type of medication (e.g., nitrate-based), as attested by a “Yes” response in the example of FIG. 3B. In some embodiments, operation 108 is performed by a processor component the same as or similar to information component 30 and/or HSA component 32.

At operation 110 of method 100, user 40 may be withheld drug 45. In some

embodiments, operation 110 is performed by a processor component the same as or similar to code component 36 (shown in FIG. 1 and described herein).

At operation 112 of method 100, whether user 40 confirms or attests a need for consultation may be determined. Responsive to the confirmation or attestation, user 40 may be provided three options. For example, if the user chooses/clicks on the telehealth option “Yes, Connect Me,” they may proceed to operation 114 whereupon a new UI screen takes them to the telehealth feature that may enable them to speak to an HCP. In this example, after clicking on that yes, the user may be taken outside of the HSA to initiate the appointment pathway for a consultation. But if user 40 chooses to speak to an HCP separately, they may receive the screen “Thank you for taking the survey,” which will inform them that they may receive communication (e.g., email) with a summary or list of instructions on what to talk to the HCP about and instructions on how to continue the HSA later. This communication may include information on a time period (e.g., 30-days) to guide consumer 40) on their HCP consultation. This service also identifies to the user that he is at risk and should consult an HCP, to address his health conditions over time and/or minimize the impact over time. Intervention may happen sooner because of the knowledge associated with this tool.

First-time users may automatically be exited out of the HSA, responsive to a yes determination at operation 112, to provide user 40 an opportunity to consult with their HCP. They may have, e.g., 30 days to schedule an appointment and confirm (e.g., at operation 116) that they have consulted with their HCP. In some embodiments, operation 112 is performed by a processor component the same as or similar to ADPBU component 34 (shown in FIG. 1 and described herein).

At operation 114 of method 100, telehealth may be seamlessly integrated, responsive to a confirmation to undergo the HCP consultation at a UI. Telehealth may be an added service into the flow; e.g., including partnership with an external group to help address any risks determined by the algorithm in the HSA. For example, telemedicine may be a key enabler to deliver against the exception-based consumer (e.g., who has ED). User 40, who did not have any DNU, ADBU, or ADPBU conditions and who are eligible to receive OTC drug 45, may be advised to speak to their HCP about their use of the product and any potential underlying conditions at their next appointment before receiving purchase code 62.

In some embodiments, user 40 may be exited out of the HSA and connected in real-time or near-real-time with a telehealth provider. For example, the user may have a telephone call, virtual call (e.g., via Zoom, Skype. Teams, or other suitable technology), or SMS/chat-based discussion to determine whether it is safe to proceed to take drug 45. In other embodiments, user 40 may be exited out of the HSA and given instructions and a link, e.g., with the instructions comprising an email providing guidance as to what the user is to talk to a doctor, pharmacist, or nurse practitioner about (e.g., health conditions x, y, and z, as depicted in FIG. 4A); the link may be subsequently used by user 40 to return to the HSA. In at least one of these embodiments, to enable a robust consumer-doctor discussion, the HSA may provide an ADPBU summary screen which may list all of the ADPBU conditions that the consumer self-reported. The telehealth provider may be a contracted and impartial third party of system 10, and said provider may comprise a licensed medical professional who may already know the reason for the telehealth visit coming through the HSA's OTC platform. User 40, who selects the telehealth feature, may also be given the ADBU/ADPBU summary to bring to the telehealth consultation for the HCP to determine if drug 45 is safe for OTC dispensing. The HCP may thus advise user 40 whether they should use drug 45 based on the consultation. The contracted telehealth service may have an administrative system that may be connected to the HSA platform, e.g., enabling the HCP at the telehealth service to validate whether the user should take drug 45. In some embodiments, operation 114 is performed by a processor component the same as or similar to ADPBU component 34 (shown in FIG. 1 and described herein) or an external third party.

At operation 116 of method 100, whether user 40 confirms safety for drug 45 may be determined. For example, user 40 may enter the HSA again and be asked “Did your Telehealth doctor determine safe use for you?” The HSA may restrict first-time users from purchasing OTC who respond “Yes” or “I don't know” to the ADBU/ADPBU warnings until they have received advice from their HCP that it is safe to use drug 45. If drug 45 is deemed safe, user 40 may be presented a UI comprising a “One Last Thing” screen and/or a screen with code 62. That is, upon returning to the HSA, user 40 may be asked if they have consulted with an HCP and if their HCP has determined that drug 45 is safe for them to purchase OTC and use. If they respond “Yes” to both questions, they may be allowed to progress to a confirmation screen. If they respond “No,” they will be taken to a UI that thanks them and facilitates an exit of the HSA.

The consumer may need to self-report a result of the consultation (e.g., their HCP's approval to use drug 45) into the HSA to receive code 62 for purchase. For example, detailed questions about nitrate medicines and a related heart problem may be asked by a physician as part of a medical assessment to ensure that if there are men prescribed nitrate medicines who have not have been screened out by the HSA, they are still denied drug 45 (e.g., by proceeding to operation 110). In some embodiments, operation 116 is performed by a processor component the same as or similar to ADPBU component 34 (shown in FIG. 1 and described herein).

At operation 118 of method 100, an unlocking code may be provided. For example, code 62 may be unlocked upon a determination that user 40 undergoing the HSA is authorized to purchase drug 45, e.g., which may be made based on a resultant, criterion-satisfying score of the user that completes at least a portion of the health survey. Code 62 may be unlocked immediately prior to a purchase. If user 40 purchases drug 45, then code 62 may enable database component 38 to track purchases over time. When purchased at kiosk 50, a range of technology solutions may be used to secure the product and only allow access to users 40 with purchase approval code 62.

Only upon confirmation or completed attestations for using product 45 and by not falsifying information, user 40 may be given code 62 to complete the OTC purchase. For example, payment may be made after the code is presented on a screen of UE 64 or sent electronically. Purchase code 62 may be valid for only a period of time (e.g., 2 weeks). As such, user 40 may have sat at their home, did the HSA, had this code, and not yet purchased until a near end of this purchasing period. Or user 40 may have just stopped by the store and decided to get to the drug's website and taken the HSA using their own UE 64 at that store and gotten code 62 to then purchase. But if the consumer is unable to complete the transaction within the purchasing period (e.g., 2 weeks), they may need to retake the HSA. The consumer purchasing online may be prompted for the purchase code. If code 62 is unavailable for use, user 40 may not be able to complete the online transaction to purchase product 45. For users at kiosk 50, an available code may be used at retail to purchase and unlock product 45 from a compartment.

In some embodiments, user 40 may obtain code 62 and provide it by uploading it online or by displaying the code to QR reader 65 of kiosk 50, as shown in the examples of FIGS. 6A and 6B. UE 64 may also implement a code reader via an on-board camera to trigger the initial opening of the HSA, i.e., upon reading code 60 of kiosk 50.

As user 40 goes through the HSA, they may get three potential outcomes in their first time through. User 40 may either be told not to use drug 45 as it is not safe for them, they may be told to ask the doctor or pharmacist, or they may get code 62 for purchase. If they get a code for purchase, database component 38 may allow them to opt into a database (e.g., storage 22), and then their path to purchase continues to checkout, e.g., where they may have three ways to buy. In some embodiments, HSA component 32 may implement web-based software such that code component 36 (e.g., after ADPBU component further confirms safety) generates code 62 to then enable an online purchase. The user may also begin the process at the kiosk, via the scan of a code, or by visiting the Cialis website to begin the HSA. Rather than using code 62, user 40 may use a fingerprint or retina scan at kiosk 50 to facilitate the unlocking of drug 45 for the person who underwent the HSA and was deemed safe. In some embodiments, operation 118 is performed by a processor component the same as or similar to code component 36.

At operation 120 of method 100, a way to complete an online purchase may be selected, via a UI from among shipping to a home and in-store pickup at locked kiosk 50. For example, user 40 may select, via a UI, the way to pay and collect from among an online purchase 120-1, shipping to a home 120-2, a kiosk at a store 120-3 having a card reader or NFC, an autonomous delivery car/truck or a drone with any suitable purchasing means, on-demand consumer delivery services (e.g., Uber Eats, DoorDash, and the like), and a vending machine outside of a store. As such, user 40 may approach a store shelf with their code and be able to unlock the product using their still-valid code and then check out. Code 62 may thus unlock the product at a shelf without needing to talk with or interface with anyone at check out. In some embodiments, operation 120 is performed at UE 64.

At operation 122 of method 100, information to the user about a need to obtain a new code may be sent (e.g., directly to UE 64 or indirectly to an account of user 40). For example, information may be provided to user 40 about getting a new code via database component 38 (shown in FIG. 1 and described herein). Since everyone who uses the OTC-enabling HSA must register, regular communication with the registered consumer may be performed via a CRM program to send emails with reminders to re-supply the product and ensure they have a valid approval code, receive healthy eating tips, encouragements to exercise, and reminders to schedule a visit with a doctor for a health checkup (e.g., as their health condition can be a sign of a more serious underlying condition). The registration process may safeguard against inappropriate access and use of drug 45. Being a registered user can also provide users to subscription access to the product and instructions on how to keep their approval code status current. And the CRM may serve to inform or remind users 40) that they must make sure that their code is active and valid for each purchase, that it may need to be reactivated. But they will be able to opt into the CRM portion, which may be what drives the messaging. In some embodiments, operation 122 is performed by a processor component the same as or similar to database component 38.

At operation 124 of method 100, a new code may be unlocked, responsive to the user answering follow-up questions with respect to any health-related changes. For example, user 40 who has already gone through all of the HSA and has purchased drug 45 may be coming back. In this example, ADPBU component 34 may not automatically send the user off to the HCP. Instead, user 40 may be asked whether a particular scenario applies to them. If so, then the user may be sent off to the HCP and may have to come back and self-report their condition. Upon having cleared through all of that, then they may go through the final leg and get a new purchase code 62. In this or another example, 3-4 questions may be asked of the user, including whether anything has changed in their health history (e.g., whether they have started any new medications and whether a dose has changed for medication they are taking) since the last time of purchase. As such, just a few short questions to make sure everything is still the same may be asked, and then they may revalidate their code for purchase. This abbreviated pathway may result in a code valid for three months, or user 40) may have to take the full survey again.

If a returning user is intending to purchase drug 45 again within a time window (e.g., three months) of the first purchase, they may be presented an abbreviated HSA. The consumer may be presented a “Welcome” screen, along with the UIs of FIGS. 2A and 2B. After this, they may be asked in the abbreviated HSA whether their health condition or medication regimen has changed. If the consumer responds “No” to both questions, they may be asked if they have talked to their doctor about potential underlying conditions, reminded to tell their doctor that they are taking drug 45 at their next appointment, and then taken to the “Important Information Screen” and “Code Screen” to obtain access to the medicine. If they respond “Yes” to either question, they may need to re-take the HSA starting from an educational message related to an underlying condition, which may be followed by questions of DNU, ADBU, ADPBU, etc. This resupply pathway may be active for three months, mimicking clinical practice for prescriptions and refills related to chronic stable conditions. As such, the addition of two screening questions provides additional safeguards for the OTC environment which are not required for refill access in the prescription environment. In some embodiments, operation 124 is performed by a processor component the same as or similar to code component 36 (shown in FIG. 1 and described herein).

At operation 126 of method 100, one or more anti-gaming measures may be provided, e.g., including counting and comparing against a threshold a number of pills of the drug purchased by the user in a determined time frame. In some embodiments, operation 126 is performed by a processor component the same as or similar to antigaming component 38 (shown in FIG. 1 and described herein).

Techniques described herein can be implemented in digital electronic circuitry, or in computer hardware, firmware, software, or in combinations of them. The techniques can be implemented as a computer program product, i.e., a computer program tangibly embodied in an information carrier, e.g., in a machine-readable storage device, in machine-readable storage medium, in a computer-readable storage device or, in computer-readable storage medium for execution by, or to control the operation of, data processing apparatus, e.g., a programmable processor, a computer, or multiple computers. A computer program can be written in any form of programming language, including compiled or interpreted languages, and it can be deployed in any form, including as a stand-alone program or as a module, component, subroutine, or other unit suitable for use in a computing environment. A computer program can be deployed to be executed on one computer or on multiple computers at one site or distributed across multiple sites and interconnected by a communication network.

Method steps of the techniques can be performed by one or more programmable processors executing a computer program to perform functions of the techniques by operating on input data and generating output. Method steps can also be performed by, and apparatus of the techniques can be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for executing instructions and one or more memory devices for storing instructions and data. Generally, a computer will also include, or be operatively coupled to receive data from or transfer data to, or both, one or more mass storage devices for storing data, such as, magnetic, magneto-optical disks, or optical disks. Information carriers suitable for embodying computer program instructions and data include all forms of non-volatile memory, including by way of example semiconductor memory devices, such as, EPROM, EEPROM, and flash memory devices: magnetic disks, such as, internal hard disks or removable disks: magneto-optical disks: and CD-ROM and DVD-ROM disks. The processor and the memory can be supplemented by, or incorporated in special purpose logic circuitry.

Several embodiments of the disclosure are specifically illustrated and/or described herein. However, it will be appreciated that modifications and variations are contemplated and within the purview of the appended claims.

The ornamental design of FIGS. 8-28 are shown with some solid lines. The broken lines, which may include words, form no part of the ornamental UI designs. The appearance of the images sequentially transition between the images shown in each of FIGS. 8-13 and FIGS. 14-19, each of FIGS. 20-28 being other, individual embodiments. The process or period in which one image transitions to another image forms no part of the ornamental design. The inventor reserves the right to claim any part. portion. element or combinations thereof of the disclosed designs. including replacing any solid line in any current or future line drawing with a broken line or vice versa.

1. A method of self-selecting provision of a drug, the method comprising:
selecting a mode of implementing a health survey preapproved by a regulatory body;
controlling, via the selected mode and a processor, a sequence of the health survey;
determining, via a processor, whether a user undergoing the survey is authorized to purchase the drug; and
responsive to the user being authorized, providing a plurality of ways to make the purchase.

2. The method of claim 1, wherein the health survey comprises a plurality of questions for assessing a medical condition, and
wherein the undergoing of the survey is performed by the user further selecting answers to the questions at one or more user interfaces, the sequence comprising a plurality of user interfaces all but an initial one of which is determined based on the further selected answer of previous question(s).

3. The method of claim 2, wherein the controlling is performed by iteratively providing (i) the initial one question on personal attributes, (ii) a question on a health-related condition, (iii) a confirmation of compliance, (iv) a question on a health-related risk, (v) a confirmation based on a determined need to consult a doctor, nurse-practitioner, or pharmacist before authorizing use of the drug, and (vi) a conditional confirmation as to whether the user consulted the doctor, nurse-practitioner, or pharmacist.

4. The method of claim 1, further comprising:
selecting, via the user at a user interface, the way to make the purchase at least from among an online purchase with shipping to a home, an online purchase with pickup at a store, on-demand consumer delivery service, and a purchase upon the user interacting with a code at a kiosk or vending machine at the store.

5. The method of claim 3, further comprising:
unlocking, via a processor, a code based on a resultant, criterion-satisfying score of the user that completes at least a portion of the health survey.

6. The method of claim 3, further comprising:
responsive to the confirmation based on the determined need for the consultation, seamlessly integrating a free telehealth session,
wherein the doctor, nurse-practitioner, or pharmacist of the telehealth session has prior knowledge of the seamless integration and the drug,

7. The method of claim 6, wherein the seamless integration is performed in real-time and includes an electronic purchase.

8. The method of claim 1, wherein the mode comprises at least one of an application-based questionnaire and a web-based questionnaire.

9. The method of claim 5, wherein the unlocking is performed by obtaining the code from secured storage or by generating a new, valid code.

10. The method of claim 1, further comprising:
initially providing a user interface such that the user is operable to register with personal information that is subsequently stored in a database.

11. The method of claim 5, wherein the unlocked code is valid only for a predetermined period of time and is linked only to the user.

12. The method of claim 11, further comprising:
sending information to the user about a need to obtain a new code for resupply or repurchase,
wherein the user has previously opted-in to receive the information.

13. The method of claim 12, further comprising:
responsive to the user answering follow-up questions with respect to any health-related changes or health care professional advice, unlocking a new code when eligible.

14. The method of claim 1, wherein one or more health risks are attributed to users who are contraindicated for the drug.

15. The method of claim 1, further comprising:
providing one or more anti-gaming measures, at least one of which comprises counting and comparing against a threshold a number of pills of the drug purchased by the user in a determined time frame.

16. A non-transitory, computer-readable medium including instructions for self-selecting provision of a drug, the instructions being executed by a processor to perform:
implementing a mode that provides a health survey preapproved by a regulatory body;
controlling, via the mode, a sequence of the health survey;
determining whether a user undergoing the survey is authorized to purchase the drug; and
responsive to the user being authorized, providing a plurality of ways to make the purchase.

17. The medium of claim 16, wherein the health survey comprises a plurality of questions for assessing a medical condition, and
wherein the undergoing of the survey is performed by the user further selecting answers to the questions at one or more user interfaces, the sequence comprising a plurality of user interfaces all but an initial one of which is determined based on the further selected answer of previous question(s).

18. The medium of claim 17, wherein the controlling is performed by iteratively providing (i) the initial one question on personal attributes, (ii) a question on a health-related condition, (iii) a confirmation of compliance, (iv) a question on a health-related risk, (v) a confirmation based on a determined need to consult a doctor, nurse-practitioner, or pharmacist before authorizing use of the drug, and (vi) a conditional confirmation as to whether the user consulted the doctor, nurse-practitioner, or pharmacist.

19. The medium of claim 16, wherein the processor further performs:
selecting, via the user at a user interface, the way to make the purchase at least from among an online purchase with shipping to a home, an online purchase with pickup at a store, and a purchase upon the user interacting with a code at a kiosk or vending machine at the store.

20. The medium of claim 18, wherein the processor further performs:
unlocking a code based on a resultant, criterion-satisfying score of the user that completes at least a portion of the health survey.
